(19) United States
(12) Patent Application Publication
(10) Pub. No.: US 2024/0192069 A1
(43) Pub. Date: Jun. 13, 2024

(54) BLOOD-PRESSURE GAUGE AND METHOD FOR MEASURING BLOOD PRESSURE

(71) Applicant: OMRON HEALTHCARE CO., LTD., Muko-shi (JP)

(72) Inventors: Yoshihiko SANO, Kyoto (JP); Kosuke ABE, Kyoto (JP)

(73) Assignee: OMRON HEALTHCARE CO., LTD., Muko-shi (JP)

(21) Appl. No.: 18/584,225

(22) Filed: Feb. 22, 2024

Related U.S. Application Data
(63) Continuation of application No. PCT/JP2022/045822, filed on Dec. 13, 2022.

(30) Foreign Application Priority Data
Dec. 28, 2021 (JP) 2021-214913

(51) Int. Cl. G01L 7/18; G01L 19/00

(52) U.S. Cl. CPC G01L 7/182; G01L 19/0023; G01L 19/0038

(57) ABSTRACT

A blood-pressure gauge includes: an opening valve connected to a sensing cuff and being in an open state with being conducted to outside air or a closed state with not being conducted to the outside air; a pressure cuff controller controlling the pressure cuff to a compression state with fluid supply to the pressure cuff and a measurement site being compressed via the pressure cuff or a release state with the fluid discharging from the pressure cuff and compression releasing via the pressure cuff; an opening valve controller controlling the opening valve to the open state or the closed state; and a blood pressure calculator calculating a blood pressure from a pressure change of the sensing cuff at the closed state of the opening valve. The sensing cuff has restorability to make a volume in the sensing cuff a predetermined volume when the opening valve is in the open state.

### CROSS-REFERENCE TO RELATED APPLICATIONS

This is a bypass continuation application of International Patent Application No. PCT/JP2022/045822 with an international filing date of Dec. 13, 2022, which claims priority of Japanese Patent Application 2021-214913 filed on Dec. 28, 2021 the entire contents of which are incorporated herein by reference.

### TECHNICAL FIELD

The present invention relates to a blood-pressure gauge and a method for measuring a blood pressure, and more particularly to a blood-pressure gauge attached to a measurement site while surrounding the measurement site in a circumferential direction, and a method for measuring a blood pressure using the blood-pressure gauge.

### BACKGROUND ART

Conventionally, as this type of blood-pressure gauge, for example, there is a blood-pressure gauge disclosed in JP 2018-102872 A. The blood-pressure gauge includes a pump, a sensing cuff in contact with a human body, and a pressure cuff that presses the sensing cuff. In this blood-pressure gauge, the sensing cuff and the pressure cuff are pressurized by the pump, and the blood pressure is calculated from pressure pulse wave information from the sensing cuff. The sensing cuff and the pressure cuff are connected to a common pump, and a switching valve that blocks air is provided between the pump and the sensing cuff.

### SUMMARY

In the conventional technique as described above, the pump, the switching valve, the pressure sensor, and the like are often built in the main body on the back of the hand, and the routing of the air path connecting from the main body to the sensing cuff on the palm side becomes complicated and long.

Therefore, an object of the present invention is to provide a blood-pressure gauge capable of simplifying the routing of an air path connected to a sensing cuff.

In order to solve the above problems, a blood-pressure gauge of this disclosure includes:
a pump;
a bag-shaped pressure cuff that is connected to the pump, and extends along a circumferential direction of a measurement site in order to received supply of a pressurizing fluid from the pump;
a sensing cuff that is not connected to the pump, includes a first sheet disposed to face an inner peripheral surface of the pressure cuff and a second sheet facing the first sheet, is configured in a bag shape, and extends in a circumferential direction so as to cross an artery passing portion of the measurement site;
a back plate that is interposed between the pressure cuff and the sensing cuff, extends along a circumferential direction of the measurement site, and transmits a pressing force from the pressure cuff to the sensing cuff;
an opening valve that is connected to the sensing cuff and is in either an open state in which an inside of the sensing cuff is conducted to outside air or a closed state in which the inside of the sensing cuff is not conducted to the outside air;
a pressure cuff controller that controls the pressure cuff to either a compression state in which a fluid is supplied to the pressure cuff and the measurement site is compressed via the pressure cuff or a release state in which the fluid is discharged from the pressure cuff and the compression of the measurement site via the pressure cuff is released;
an opening valve controller that controls the opening valve to be in either the open state or the closed state; and
a blood pressure calculator that calculates a blood pressure on a basis of a pressure change of the sensing cuff when the opening valve is in the closed state, in which
the sensing cuff has restorability to make a volume in the sensing cuff a predetermined volume when the opening valve is in the open state.

The “fluid” is typically air, but may be other gases or liquids.

The “inner peripheral side” of the pressure cuff refers to a side facing the measurement site in an attachment state surrounding the measurement site.

In the blood-pressure gauge of the present disclosure, the inside of the sensing cuff is conducted to the outside air when the opening valve is in the open state, and is not conducted to the outside air when the opening valve is in the closed state. The sensing cuff has restorability so that the volume in the sensing cuff is a predetermined volume when the opening valve is in the open state. Therefore, the volume in the sensing cuff can be set to a predetermined volume only by opening the opening valve, restoring the sensing cuff, and closing the opening valve without routing the flow path from the means such as the pump that supplies the fluid to the sensing cuff from the pump side to the sensing cuff side. Thereafter, the pressure cuff controller supplies the fluid to the pressure cuff to bring the pressure cuff into the compression state of compressing the measurement site via the pressure cuff, and when the opening valve is in the closed state, the blood pressure is calculated based on the pressure change of the sensing cuff. Therefore, the air path connecting from the pump side to the sensing cuff side becomes unnecessary, and the routing of the air path connected to the sensing cuff can be simplified.

### BRIEF DESCRIPTION OF THE DRAWINGS

FIG. 1 is a front view illustrating a schematic appearance configuration of a blood-pressure gauge according to a first embodiment;

FIG. 2 is a side view illustrating a schematic appearance configuration of the blood-pressure gauge according to the first embodiment;

FIG. 3 is a perspective view illustrating a schematic appearance configuration of the blood-pressure gauge according to the first embodiment;

FIG. 4 is a cross-sectional view illustrating a state in which the blood-pressure gauge according to the first embodiment is attached to the wrist;

FIG. 5A is a partially omitted planar layout when a back plate and a sensing cuff in a cuff structure of the blood-pressure gauge according to the first embodiment are in an unfolded state with a surface of the back plate facing a curler as a foremost surface;

FIG. 5B is a cross section taken along line B-B′ in FIG. 5A;

FIG. 5C is a cross section taken along line A-A′ in FIG. 5A;

FIG. 6 is an enlarged view of FIG. 5C;

FIG. 7 is a diagram illustrating a schematic configuration related to a flow path system of the blood-pressure gauge according to the first embodiment;

FIG. 8 is a diagram illustrating a schematic configuration related to a control system of the blood-pressure gauge according to the first embodiment;

FIGS. 9A to 9C are cross-sectional views of a pressure cuff, a back plate, and a sensing cuff along a direction in which an artery of a subject extends;

FIG. 10 is a flowchart illustrating an operation in the blood-pressure gauge according to the first embodiment;

FIG. 11 is a schematic configuration diagram related to a flow path system of the blood-pressure gauge for explaining the operation of the blood-pressure gauge according to the first embodiment;

FIG. 12 is a schematic configuration diagram related to a flow path system of the blood-pressure gauge for explaining the operation of the blood-pressure gauge according to the first embodiment;

FIG. 13 is a schematic configuration diagram related to a flow path system of the blood-pressure gauge for explaining the operation of the blood-pressure gauge according to the first embodiment;

FIG. 14 is a schematic configuration diagram related to a flow path system of the blood-pressure gauge for explaining the operation of the blood-pressure gauge according to the first embodiment;

FIG. 15 is a cross-sectional view of a sensing cuff, a nipple portion, and an elastic member in a modification;

FIG. 16 is a cross-sectional view of a sensing cuff, a nipple portion, and an elastic member in a modification;

FIG. 17 is a cross-sectional view of a sensing cuff, a nipple portion, and an elastic member according to a second embodiment; and

FIG. 18 is a cross-sectional view of the sensing cuff, the nipple portion, and the elastic member according to the second embodiment.

### DETAILED DESCRIPTION

### First Embodiment

Hereinafter, a first embodiment of the present invention will be described in detail with reference to the drawings.

### (Configuration of Blood-Pressure Gauge)

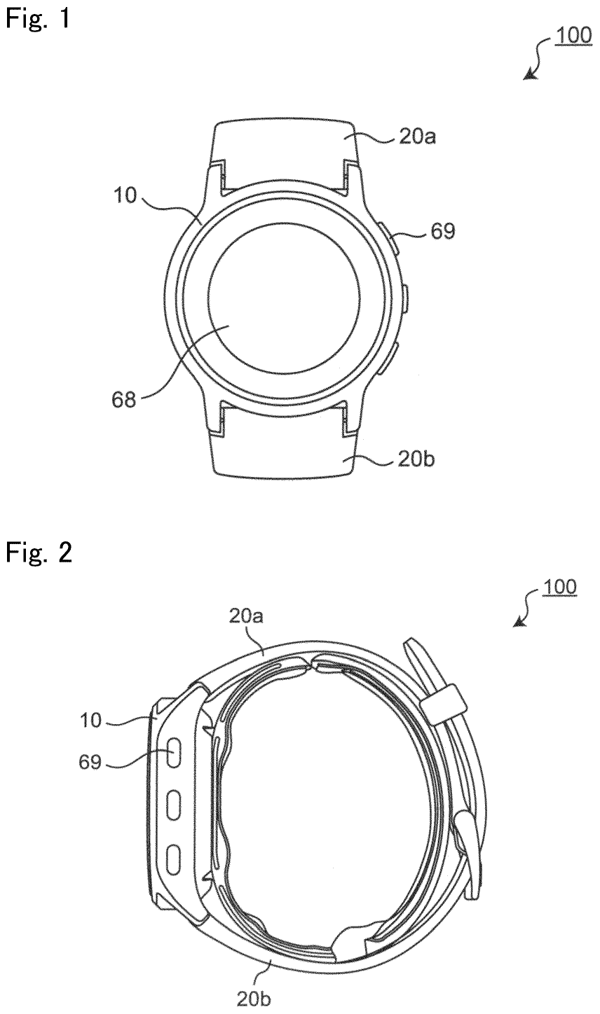

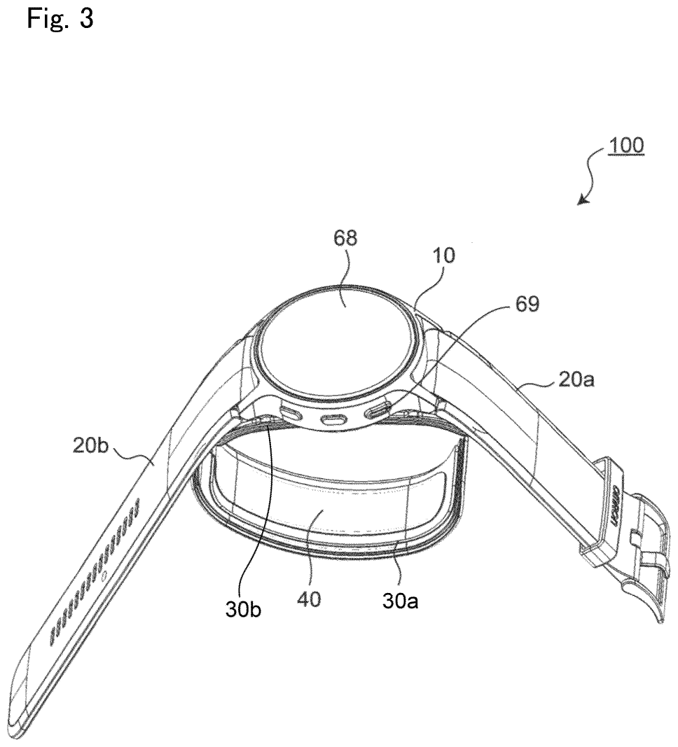

FIG. 1 illustrates a configuration of a blood-pressure gauge 100 according to the present embodiment as viewed from the front. FIG. 2 illustrates a configuration of the blood-pressure gauge 100 as viewed from a side surface. FIG. 3 illustrates a configuration of the blood-pressure gauge 100 as viewed from an oblique direction in a state where a belt described later is opened. A schematic appearance configuration of the blood-pressure gauge 100 will be described with reference to FIGS. 1 to 3.

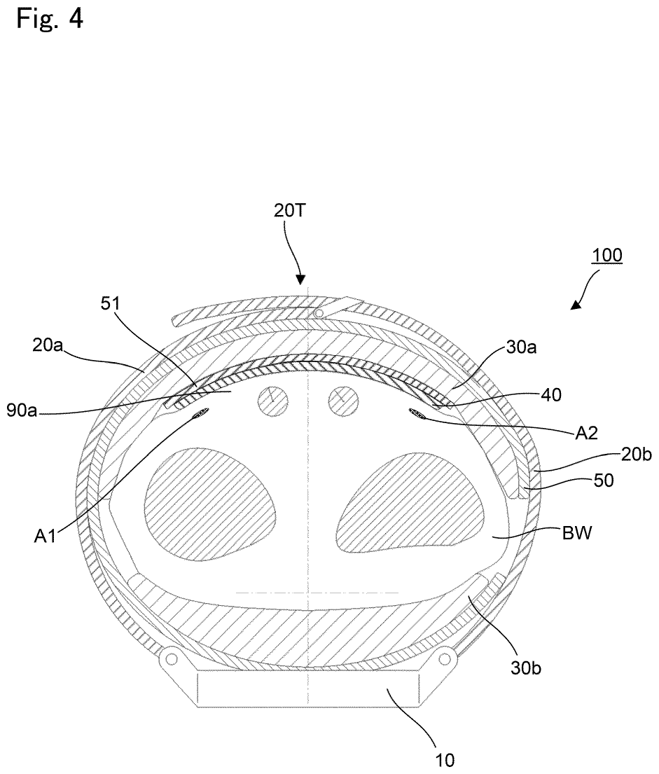

The blood-pressure gauge 100 includes a main body 10 and two belts 20a, 20b extending from the main body 10 and attached around a measurement site (In this example, as illustrated in FIG. 4 to be described later, a left wrist BW is scheduled as the measurement site.). By fastening the one belt 20a and the other belt 20b, a state in which the blood-pressure gauge 100 is attached to the measurement site (See FIG. 4, which is referred to as an “attachment state”) is created. Furthermore, as illustrated in FIGS. 1 to 3, the main body 10 includes a display device 68 and an operation device 69 including a plurality of buttons. Furthermore, the main body 10 is mounted with a pump to be described later.

As illustrated in FIG. 3, the blood-pressure gauge 100 includes pressure cuffs 30a, 30b and a sensing cuff 40. The pressure cuff 30a is a pressure cuff located on the measurement site side close to the artery, and the pressure cuff 30b is a pressure cuff located on the main body 10 side opposite to the measurement site side.

In the present embodiment, the pressure cuffs 30a, 30b and the sensing cuff 40 constitute a cuff structure having a laminated structure. In the attachment state of the blood-pressure gauge 100, the pressure cuff 30a and the sensing cuff 40 are arranged in this order when viewed from a fastening portion 20T side of the belts 20a, 20b. The pressure cuff 30b is disposed on the main body 10 side.

As illustrated in FIG. 4, the cuff structure according to the present embodiment further includes a curler 50 and a back plate 51. The curler 50 is, for example, a member made of a resin plate having a certain degree of flexibility and hardness and having a shape curved along the circumferential direction surrounding the measurement site in a natural state. The pressure cuff 30a is disposed on an inner peripheral side of the curler 50 and on a side corresponding to the measurement site, and the pressure cuff 30b is disposed on the inner peripheral side of the curler 50 and on a side close to the main body 10 on the side opposite to the measurement site. In addition, the cuff structure includes the back plate 51 between the pressure cuff 30a and the sensing cuff 40. A member including the belts 20a, 20b, the curler 50, the pressure cuffs 30a, 30b, and the back plate 51 functions as a pressure member that generates a pressure force on the measurement site. The pressure member including the pressure cuffs 30a, 30b presses the sensing cuff 40 toward the measurement site and causes the sensing cuff 40 to compress (press) the measurement site.

FIG. 4 is a cross-sectional view illustrating a state in which the blood-pressure gauge 100 is attached to the wrist BW, which is a measurement site. As illustrated in FIG. 4, the pressure cuff 30a constituting the pressure member has a bag shape and is disposed between the belts 20a, 20b and the sensing cuff 40. The pressure cuff 30b also has a bag shape, and is disposed at a position opposite to the pressure cuff 30a so as to sandwich the wrist BW between the pressure cuff 30a and the pressure cuff 30b.

As described above, the belts 20a, 20b surround the wrist BW in the circumferential direction, whereby the blood-pressure gauge 100 is attached to the wrist BW. In the attachment state of the present embodiment, as illustrated in FIG. 4, the curler 50, the pressure cuff 30b, the wrist BW, the sensing cuff 40, the back plate 51, and the pressure cuff 30a are arranged in this order from the main body 10 toward the fastening portions 20T of the belts 20a, 20b. In the configuration example of FIG. 4, the main body 10 is disposed at a portion opposite to the sensing cuff 40 in the circumferential direction of the belts 20a, 20b.

In the attachment state, the bag-shaped pressure cuffs 30a, 30b extend, for example, along the circumferential direction of the wrist BW. In addition, the bag-shaped sensing cuff 40 is disposed on the inner circumferential side of the belts 20a, 20b with respect to the pressure cuff 30a, is in contact with (indirectly or directly) the wrist BW, and extends in the circumferential direction so as to cross an artery passing portion 90a of the wrist BW. The “inner circumferential side” of the belts 20a, 20b refers to a side facing the wrist BW in the attachment state surrounding the wrist BW.

FIG. 4 illustrates a radial artery A1 and an ulnar artery A2 of the wrist BW. The pressure cuffs 30a, 30b constituting the pressure member press the sensing cuff 40 toward the wrist BW to cause the sensing cuff 40 to compress the wrist BW.

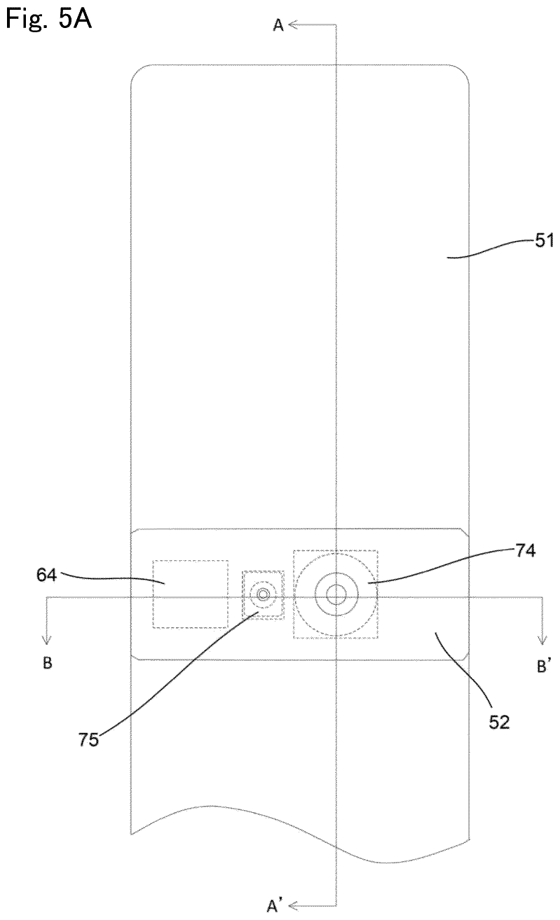

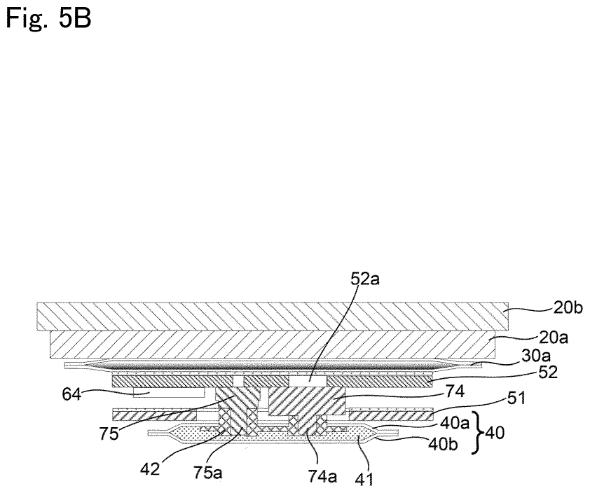

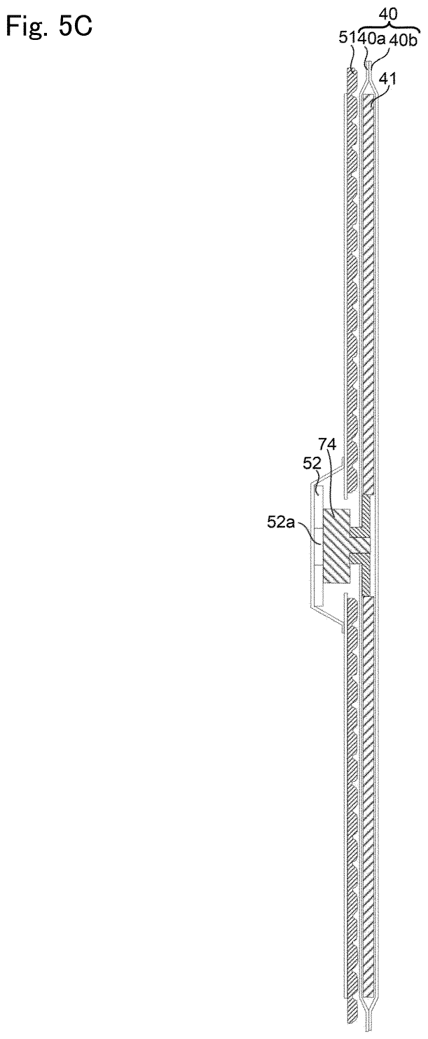

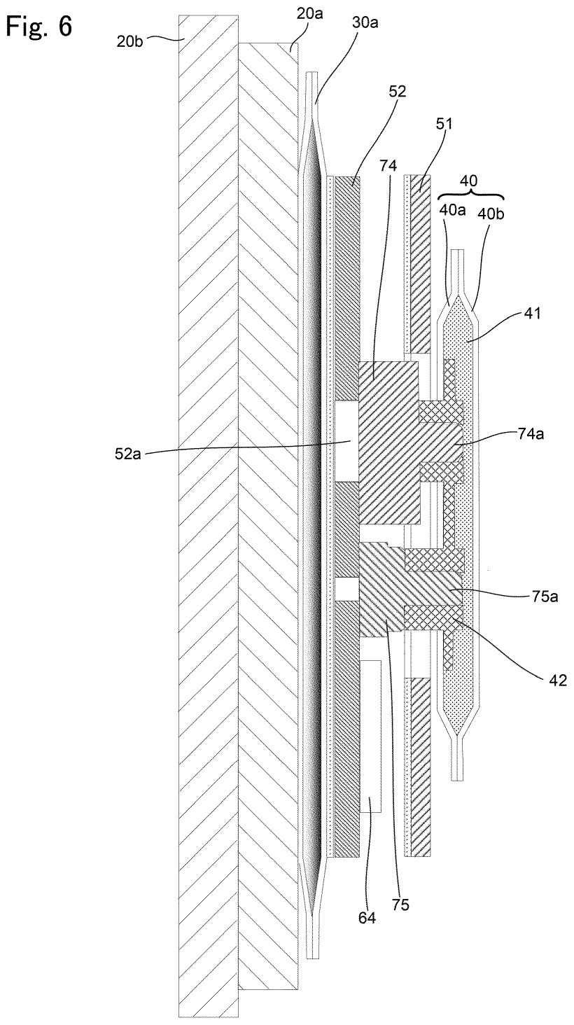

FIG. 5A is a partially omitted planar layout when the back plate 51 and the sensing cuff 40 in the cuff structure are in an unfolded state with the surface of the back plate 51 facing the curler 50 as the foremost surface. FIG. 5B illustrates a cross section taken along line B-B′ in FIG. 5A. FIG. 5C illustrates a cross section taken along line A-A′ in FIG. 5A. FIG. 5C also illustrates the pressure cuff 30a and the belts 20a, 20b. FIG. 6 is an enlarged view of FIG. 5C.

As illustrated in FIGS. 5B, 5C, and 6, the sensing cuff 40 includes a first sheet 40a disposed to face the inner peripheral surface of the pressure cuff 30a and a second sheet 40b facing the first sheet 40a. The sensing cuff 40 is formed in a bag shape in which peripheral portions of the first sheet 40a and the second sheet 40b are brought into close contact with each other by welding.

As illustrated in FIGS. 5B, 5C, and 6, an elastic member 41 is accommodated in the sensing cuff 40. The elastic member 41 has restorability so that the volume in the sensing cuff 40 becomes a predetermined volume when the opening valve 74 described later is opened and the pressure in the sensing cuff 40 is released to the atmospheric pressure. In the present embodiment, as an example, a sponge having an open-cell structure is used as the elastic member 41. In the sponge having an open-cell structure, bubbles are connected, and gas and liquid can pass through the inside of the sponge. The elastic member 41 of such a sponge is pressed and compressed at the time of blood pressure measurement, and enables pulse pressure measurement by the sensing cuff 40. However, when the pressure in the sensing cuff 40 is released to the atmospheric pressure before or after the blood pressure measurement, the elastic member 41 again contains air and is restored, and the volume of the sensing cuff 40 can be made constant. In the present embodiment, as described above, the blood pressure measurement is performed by the constant volume sensing method in which the volume in the sensing cuff 40 is set to a predetermined volume (constant volume) using the elastic member 41. Details of the blood pressure measurement by the constant volume sensing method will be described later.

As illustrated in FIGS. 5B, 5C, and 6, the opening valve 74 is mounted on a substrate 52 disposed between the pressure cuff 30a and the back plate 51. In the present embodiment, as an example, a solenoid type opening valve 74 is used. On the substrate 52 on which the opening valve 74 is mounted, an opening 52a is formed at a position corresponding to a valve port of the opening valve 74. A protruding portion 74a is provided on a surface opposite to the mounting surface with respect to the opening valve 74 substrate 52. As illustrated in FIGS. 5B, 5C, and 6, the protruding portion 74a is interposed in a nipple portion 42 fixed to the sensing cuff 40. The opening valve 74 is set to either an open state or a closed state under the control of a sub CPU 64 (to be described later) mounted on the substrate 52. When the opening valve 74 is in the open state, the valve ports on the protruding portion 74a side and the mounting surface side with respect to the substrate 52 are opened, the inside of the sensing cuff 40 is conducted to the outside air, and the pressure inside the sensing cuff 40 is released to the atmospheric pressure. When the opening valve 74 is in the closed state, the valve ports on the protruding portion 74a side and the mounting surface side with respect to the substrate 52 are closed, and the inside of the sensing cuff 40 is not conducted to the outside air.

As illustrated in FIGS. 5B, 5C, and 6, a first pressure sensor 75 for detecting the pressure of the sensing cuff 40 is mounted on the substrate 52. In this example, the first pressure sensor 75 is a piezoresistive pressure sensor. A protruding portion 75a is provided on a surface opposite to a surface on which the first pressure sensor 75 is mounted with respect to the substrate 52. As illustrated in FIGS. 5B, 5C, and 6, the protruding portion 75a is interposed in the nipple portion 42 fixed to the sensing cuff 40.

Further, as illustrated in FIGS. 5B, 5C, and 6, the sub CPU 64 is mounted on the substrate 52. The sub CPU 64 controls the open state and the closed state of the opening valve 74 and detects the pressure of the sensing cuff 40 using the first pressure sensor 75. A main CPU 65 described later mainly controls the operation of the entire blood-pressure gauge 100.

As described above, in the present embodiment, the opening valve 74, the first pressure sensor 75, and the sub CPU 64 are configured as a substrate-integrated type mounted on the substrate 52 close to the sensing cuff 40.

As illustrated in FIGS. 5B, 5C, and 6, the back plate 51 is interposed between the pressure cuff 30a and the sensing cuff 40. The back plate 51 is formed of, for example, a plate-like resin (In this example, polypropylene) having a thickness of about 1 mm, extends along the circumferential direction of the measurement site, and has a function of transmitting the pressure force from the pressure cuffs 30a, 30b to the sensing cuff 40.

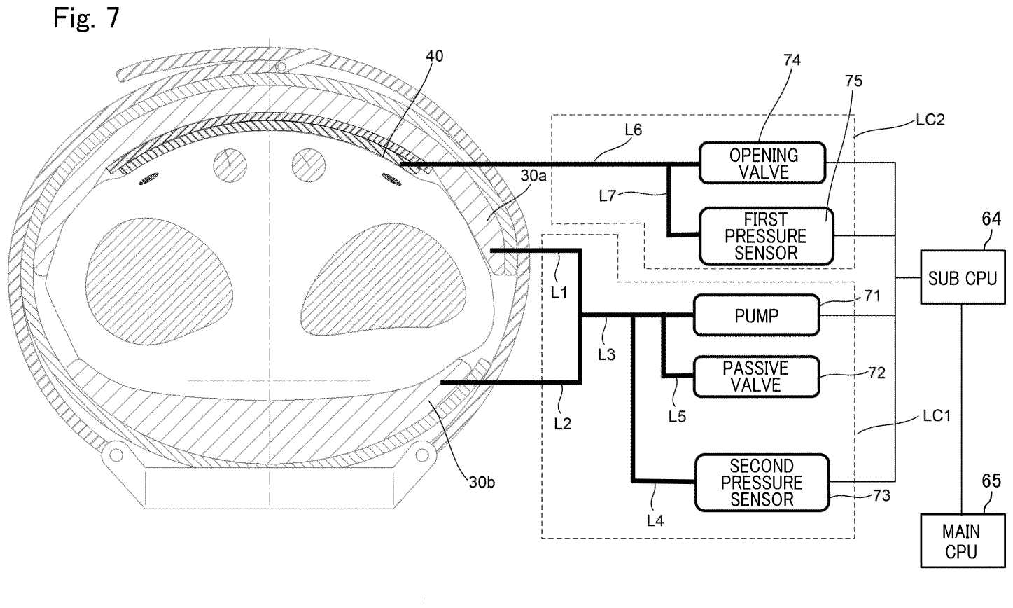

FIG. 7 illustrates a schematic configuration of a flow path system of the blood-pressure gauge 100. As illustrated in FIG. 7, the flow path system of the blood-pressure gauge 100 includes a fluid circuit LC1 connected to the pressure cuffs 30a, 30b and a fluid circuit LC2 connected to the sensing cuff 40.

The fluid circuit LC1 includes a pump 71, a passive valve 72, a second pressure sensor 73, and flow paths L1 to L5. Air circulates in each of the flow paths L1 to L5. In the fluid circuit LC1, air is supplied to the pressure cuffs 30a, 30b to be inflated or air is discharged from the pressure cuffs 30a, 30b according to on/off (supply/stop of air) of the pump 71 under the control of the sub CPU 64. When the pressure cuffs 30a, 30b are inflated, the pump 71 is turned on by the control of the sub CPU 64, air is supplied from the pump 71 to the pressure cuffs 30a, 30b via the flow paths L3, L1, and L2, and the pressure in the pressure cuffs 30a, 30b is detected by the second pressure sensor 73 and the sub CPU 64 via the flow path L4. At this time, since the passive valve 72 is pressurized via the flow path L5, it functions as a check valve, and the air in the pressure cuffs 30a, 30b is not discharged to the outside via the flow path L5. On the other hand, when the air is discharged from the pressure cuffs 30a, 30b, the pump 71 is turned off by the control of the sub CPU 64, and the passive valve 72 is not pressurized via the flow path L5. Therefore, the air in the pressure cuffs 30a, 30b is discharged from the passive valve 72 via the flow paths L1, L2, L3, and L5, and the pressure in the pressure cuffs 30a, 30b is released to the atmospheric pressure.

The fluid circuit LC2 includes an opening valve 74, a first pressure sensor 75, and flow paths L6 to L7. Air circulates in each of the flow paths L6 to L7. In the fluid circuit LC2, the air in the sensing cuff 40 is discharged or the discharge of the air from the sensing cuff 40 is prevented according to the off/on (opening/closing of the valve) of the opening valve 74 by the control of the sub CPU 64. When the air in the sensing cuff 40 is discharged, the opening valve 74 is turned off (opened state) by the control of the sub CPU 64, the air in the sensing cuff 40 is discharged through the flow paths L6, L7 and the opening valve 74, and the pressure in the sensing cuff 40 is released to the atmospheric pressure. On the other hand, when the discharge of the air from the sensing cuff 40 is prevented, the opening valve 74 is set to the ON state (closed state) by the control of the sub CPU 64, and the discharge of the air from the sensing cuff 40 through the flow paths L6, L7 and the opening valve 74 is prevented. When the opening valve 74 becomes the ON state (closed state), a change in pressure in the sensing cuff 40 is detected by the first pressure sensor and the sub CPU 64 via the flow paths L6, L7, and blood pressure measurement becomes possible.

As described above, in the present embodiment, the fluid circuit LC2 and the fluid circuit LC1 are not in the connection relationship via the flow path, and it is not necessary to route the flow path, which is the air path from the pump 71 or the like built in the main body 10 located on the back of the hand side at the time of blood pressure measurement, with respect to the sensing cuff 40 located on the palm side at the time of blood pressure measurement.

The sub CPU 64 controls the pump 71 and the opening valve 74, detects the pressure in the pressure cuffs 30a, 30b using the second pressure sensor 73, and detects the pressure in the sensing cuff 40 using the first pressure sensor 75. The opening valve 74, the first pressure sensor 75, and the sub CPU 64 are configured as a substrate integrated type integrally mounted on the substrate 52, and the sub CPU 64 and the pump 71 and the second pressure sensor 73 on the fluid circuit LC1 side can be electrically connected by a simple wiring. Therefore, in the present embodiment, it is not necessary to perform complicated wiring not only for routing the flow path which is the air path but also for electrical connection. The main CPU 65 built in the main body 10 is a CPU that mainly controls the operation of the entire blood-pressure gauge 100, and is configured to be able to communicate with the sub CPU 64. The electrical connection between the main CPU 65 and the sub CPU 64 does not need complicated wiring.

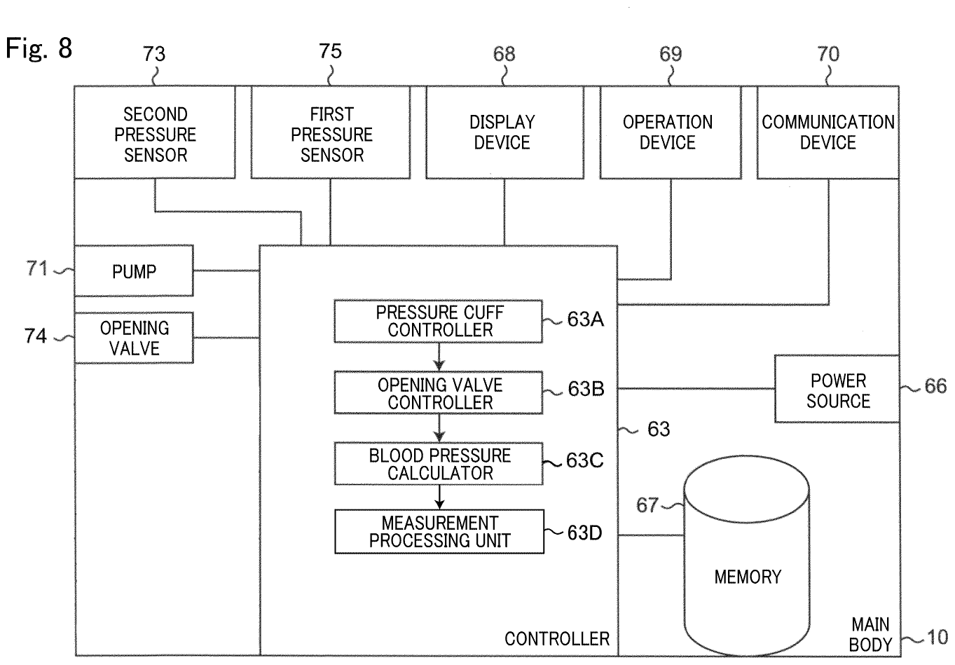

FIG. 8 illustrates a schematic configuration related to a control system of the blood-pressure gauge 100. As illustrated in FIG. 8, the main body 10 of the blood-pressure gauge 100 includes a controller 63 that performs control, and a plurality of controlled components 67 to 75 controlled by the controller 63.

In FIG. 8, the sub CPU 64 and the main CPU 65 are collectively referred to as a controller 63. In addition, the plurality of controlled components include a power source 66, a memory 67, a display device 68, an operation device 69, a communication device 70, a pump 71, a second pressure sensor (pressure cuff pressure sensor) 73, an opening valve 74, and a first pressure sensor (sensing cuff pressure sensor) 75.

In this example, the power source 66 is made of a rechargeable secondary battery. The power source 66 supplies power for driving to elements mounted on the main body 10, for example, the controller 63, the memory 67, the display device 68, the communication device 70, the pump 71, the second pressure sensor 73, the opening valve 74, and the first pressure sensor 75.

The memory 67 stores various kinds of data. For example, the memory 67 can store measurement values measured by the blood-pressure gauge 100, measurement results of the second pressure sensor 73 and the first pressure sensor 75, and the like. The memory 67 can also store various kinds of data generated by the controller 63. The memory 67 includes a random access memory (RAM), a read only memory (ROM), and the like. For example, various programs are changeably stored in the memory 67.

The display device 68 is made of, for example, a liquid crystal display (LCD). The display device 68 displays information related to blood pressure measurement such as a blood pressure measurement result and other information according to a control signal from the controller 63. Note that the display device 68 may have a function as a touch panel.

The operation device 69 includes a plurality of buttons that receive an instruction from the user. When the operation device 69 receives an instruction from the user, an operation/operation according to the instruction is performed under the control of the controller 63. Note that the operation device 69 may be, for example, a pressure-sensitive (resistance) or proximity (capacitance) touch panel switch. In addition, a configuration in which a microphone (not illustrated) is provided to receive an instruction by a user's voice may be adopted.

The communication device 70 transmits various kinds of data and various signals to an external device via a communication network, and receives information from the external device via the communication network. The network may be wireless communication or wired communication.

In this example, the pump 71 is a piezoelectric pump, and is driven on the basis of a control signal given from the controller 63. The pump 71 can supply the pressurizing fluid to the pressure cuffs 30a, 30b through flow paths described later. Here, any liquid or any gas can be employed as the fluid. In the present embodiment, the fluid is air (Hereinafter, the fluid is described as air.).

The second pressure sensor 73 and the first pressure sensor 75 are, for example, piezoresistive pressure sensors. The second pressure sensor 73 detects the pressure in the pressure cuffs 30a, 30b via the flow path L4 illustrated in FIG. 7. The first pressure sensor 75 detects the pressure in the sensing cuff 40 via the flow path L7 illustrated in FIG. 7.

The passive valve 72 is controlled according to the operation of the pump 71. That is, opening and closing of the passive valve 72 is controlled according to on/off of the pump 71 (supply/stop of air). For example, the passive valve 72 closes when the pump 71 is turned on. On the other hand, the passive valve 72 opens when the pump 71 is turned off.

The opening valve 74 is connected to the flow path L6 illustrated in FIG. 7, and is controlled to either an open state or a closed state on the basis of a control signal given from the sub CPU 64 as the controller 63. When the opening valve 74 is in the off state and in the open state, the air in the sensing cuff 40 is discharged from the opening valve 74 through the flow path L6, and the pressure in the sensing cuff 40 is released to the atmospheric pressure. On the other hand, when the opening valve 74 is in the off state and in the closed state, the discharge of air from the opening valve 74 is prevented.

In this example, the controller 63 includes a sub central processing unit (CPU) 64 and a main CPU 65. For example, the controller 63 reads each program and each data stored in the memory 67. Furthermore, the controller 63 controls each of units 67 to 75 according to the read program to execute a predetermined operation (function). In addition, the controller 63 performs predetermined calculation, analysis, processing, and the like in the controller 63 according to the read program. Note that some or all of the functions executed by the controller 63 may be configured as hardware by one or a plurality of integrated circuits or the like.

As illustrated in FIG. 8, the controller 63 according to the present embodiment includes a pressure cuff controller 63A, an opening valve controller 63B, a blood pressure calculator 63C, and a measurement processing unit 63D as functional blocks. The functions of the pressure cuff controller 63A, the opening valve controller 63B, the blood pressure calculator 63C, and the measurement processing unit 63D will be described in detail in the description of the operation to be described later.

### (Measurement Principle of Constant Volume Sensing Method)

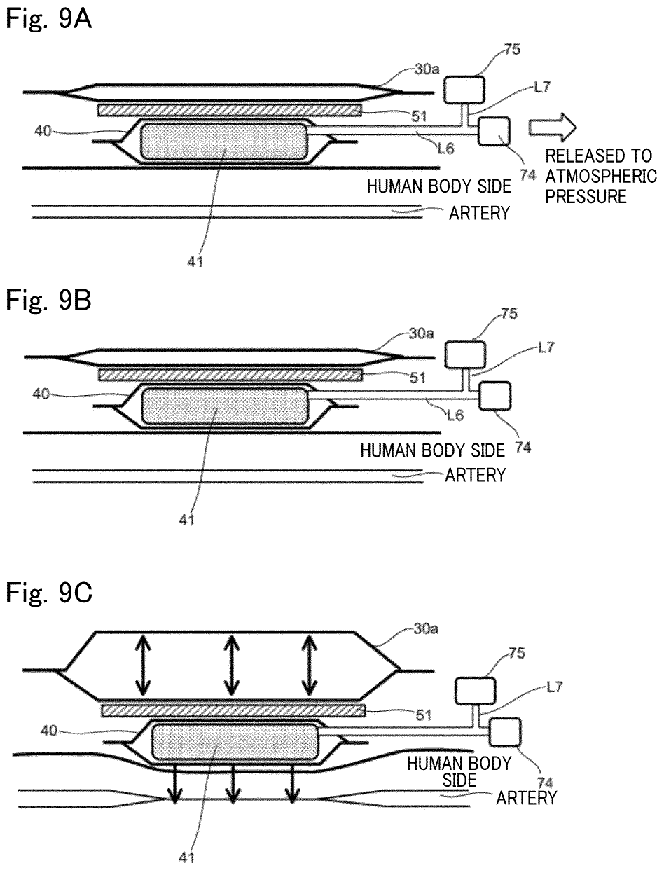

Next, the measurement principle of the constant volume sensing method in the present embodiment will be described with reference to FIGS. 9A to 9C. FIGS. 9A to 9C are cross-sectional views of the pressure cuffs 30a, 30b, the back plate 51, and the sensing cuff 40 along the direction in which the artery of the subject extends, and are views for explaining the measurement principle of the constant volume sensing method in the present embodiment.

FIG. 9A is a diagram illustrating a state of a first stage before blood pressure measurement. As illustrated in FIG. 9A, in the first stage before blood pressure measurement, the pressure cuffs 30a, 30b are in a non-pressurized state. In this state, the opening valve 74 is turned off by the control of the sub CPU 64. As a result, the opening valve 74 is opened, the air in the sensing cuff 40 is discharged from the opening valve 74 through the flow path L6, and the pressure in the sensing cuff 40 is released to the atmospheric pressure.

In the present embodiment, a sponge having an open-cell structure is disposed as the elastic member 41 inside the sensing cuff 40. Therefore, in a state in which the sensing cuff 40 does not receive the pressure force from the pressure cuffs 30a, 30b and in a state in which the pressure in the sensing cuff 40 is released to the atmospheric pressure, the elastic member 41 restores the volume in the sensing cuff 40 to a predetermined volume.

FIG. 9B is a diagram illustrating a state of a second stage before blood pressure measurement. As illustrated in FIG. 9B, in the second stage before blood pressure measurement, the pressure cuffs 30a, 30b are in a non-pressurized state. In this state, the opening valve 74 is turned on by the control of the sub CPU 64. As a result, the opening valve 74 is closed, the air in the sensing cuff 40 is prevented from being discharged from the opening valve 74 through the flow path L6, and the sensing cuff 40 is maintained at the predetermined volume.

FIG. 9C is a diagram illustrating a state at the time of blood pressure measurement. As illustrated in FIG. 9C, at the time of blood pressure measurement, the pump 71 is driven by the control of the sub CPU 64, air is supplied to the pressure cuffs 30a, 30b, and the pressure cuffs 30a, 30b and the back plate 51 pressurize the sensing cuff 40. As a result, the sensing cuff 40 is pressed against the human body, and a pressure pulse wave is detected by the first pressure sensor 75 and the sub CPU 64 as a change in pressure in the sensing cuff 40.

As described above, in the present embodiment, the sensing cuff 40 is not adjusted to the predetermined volume by the supply of air from the pump 71, but the pressure in the sensing cuff 40 is released to the atmospheric pressure by opening the opening valve 74, and the sensing cuff 40 is set to the predetermined volume by the restoring force of the elastic member 41 and then closing the opening valve 74.

As a result, in the present embodiment, it is not necessary to route the flow path, which is the air path from the pump 71 built in the main body 10, to the sensing cuff 40 side, and it is not necessary to dispose the pressure sensor for the sensing cuff 40 on the pump 71 side. Therefore, it is not necessary to route the flow path, which is the air path for the pressure sensor for the sensing cuff 40. Therefore, it is possible to simplify the routing of the flow path.

### (Operation of Blood-Pressure Gauge)

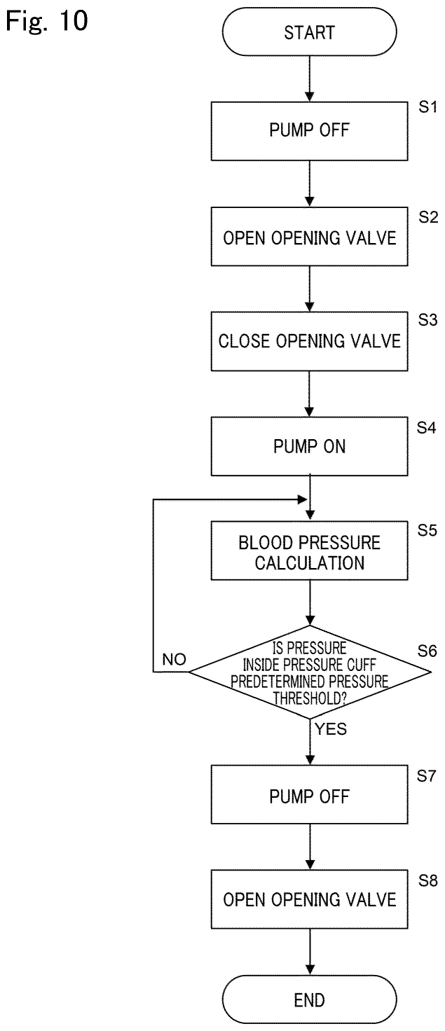

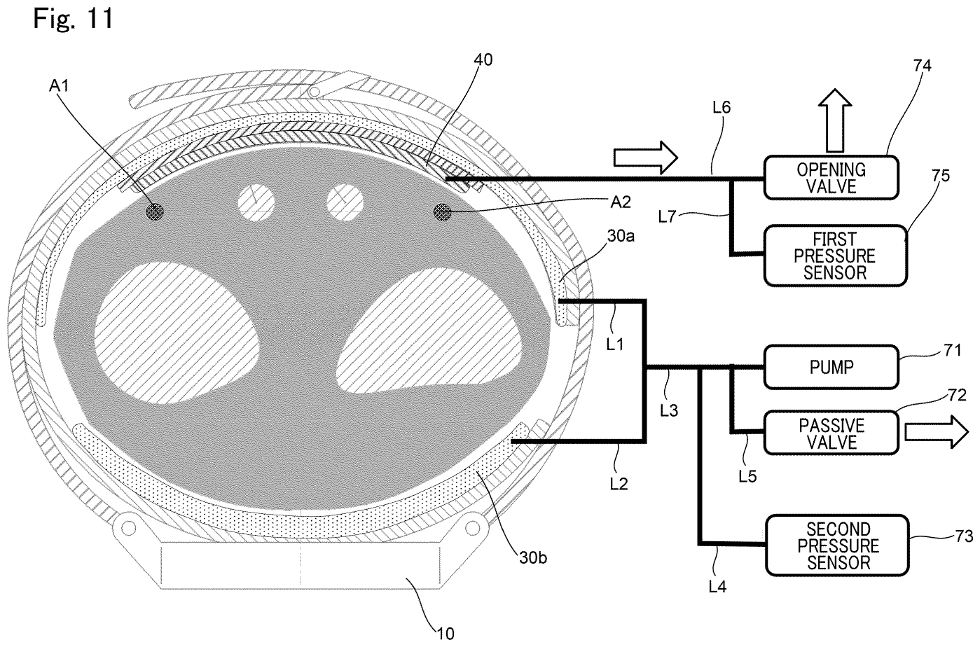

FIG. 10 is a flowchart illustrating a flow of a method for measuring a blood pressure using the blood-pressure gauge 100 according to the present embodiment. FIGS. 11 to 14 are schematic configuration diagrams regarding a flow path system of the blood-pressure gauge 100 for explaining states of the pressure cuffs 30a, 30b and the sensing cuff 40 according to the operation of the blood-pressure gauge.

First, after the blood-pressure gauge 100 is attached to the wrist BW, first-stage processing of blood pressure measurement preparation is performed. In the first-stage processing of the blood pressure measurement preparation, as illustrated in FIG. 10, in a state where the blood-pressure gauge 100 is attached to the wrist BW, the sub CPU 64 of the controller 63 turns off the pump 71 of the fluid circuit LC1 (FIG. 10: S1). As a result, as indicated by a white arrow in FIG. 11, the air in the pressure cuffs 30a, 30b is discharged from the passive valve 72, and the pressure cuffs 30a, 30b are in a non-pressurized state.

On the other hand, the sub CPU 64 of the controller 63 functions as the opening valve controller 63B, sets the opening valve 74 to the OFF state, and sets the opening valve 74 to the open state (FIG. 10: S2). As a result, the air in the sensing cuff 40 is discharged from the opening valve 74 as indicated by a white arrow in FIG. 11, and the pressure in the sensing cuff 40 is released to the atmospheric pressure. At this time, the sensing cuff 40 is restored so that the sensing cuff 40 has a predetermined volume.

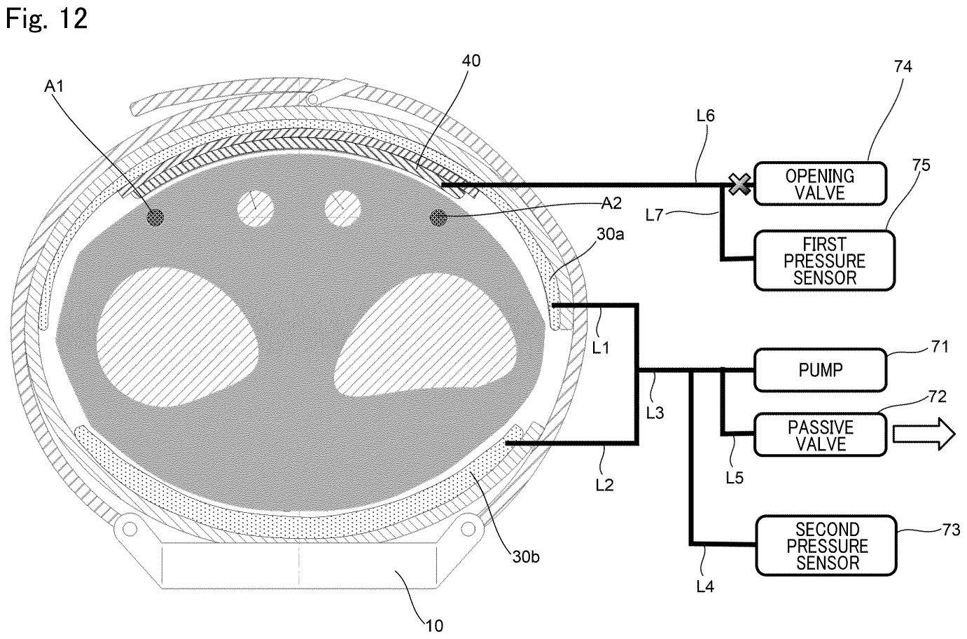

Next, second stage processing of blood pressure measurement preparation is performed. In the second stage processing of blood pressure measurement preparation, the sub CPU 64 of the controller 63 functions as the opening valve controller 63B, and turns on the opening valve 74 and closes the opening valve 74 as illustrated in FIG. 10 (FIG. 10: S3). As a result, the sensing cuff 40 is closed, and the air in the sensing cuff 40 is prevented from being discharged from the opening valve 74. A cross mark in FIG. 12 indicates that the discharge of air from the opening valve 74 is prevented.

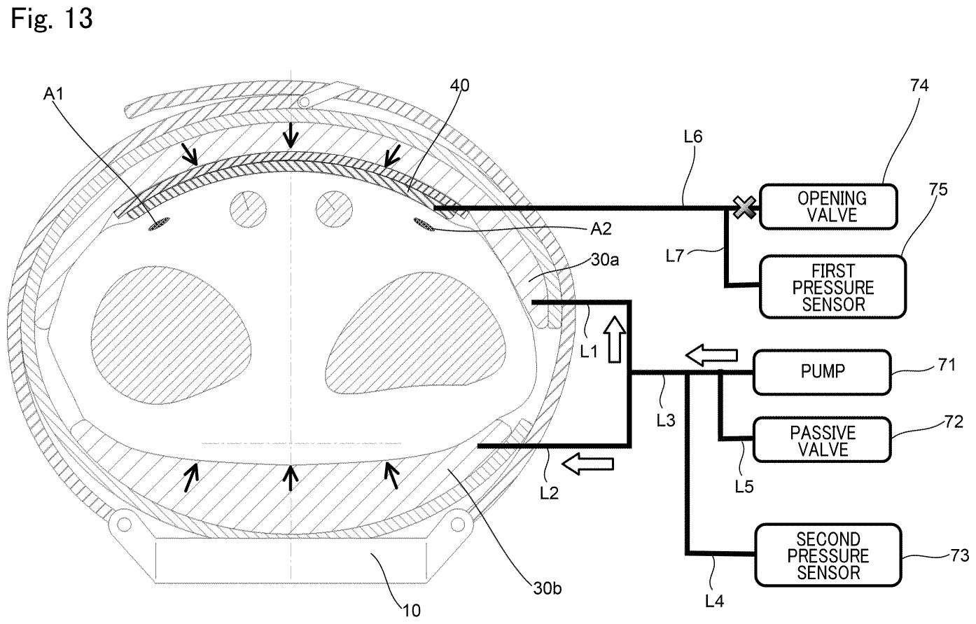

Next, processing at the time of blood pressure measurement is performed. In the processing at the time of blood pressure measurement, as illustrated in FIG. 10, the sub CPU 64 of the controller 63 functions as the pressure cuff controller 63A and turns on the pump 71 (FIG. 10: S4). As a result, as indicated by a white arrow in FIG. 13, the air is supplied from the pump 71 to the pressure cuffs 30a, 30b through the flow paths L3, L1, and L2, and the pressure cuffs are gradually pressurized.

In this manner, the sensing cuff 40 is pressed by the pressure cuffs 30a, 30b and the back plate 51 and compressed against the human body (wrist). That is, the sub CPU 64 of the controller 63 functioning as the pressure cuff controller 63A supplies air as a fluid to the pressure cuffs 30a, 30b, and controls the pressure cuffs 30a, 30b so as to bring the pressure cuffs 30a, 30b into a compression state of compressing the human body (wrist) as the measurement site via the pressure cuffs 30a, 30b. When the sensing cuff 40 is compressed against the human body (wrist), a human body pressure pulse wave is generated in the sensing cuff 40 as a pressure change of the sensing cuff 40. Then, the sub CPU 64 of the controller 63 functions as the blood pressure calculator 63C, detects the human body pressure pulse wave as the pressure change of the sensing cuff 40 by the first pressure sensor 75, and calculates the hand blood pressure by the oscillometric method (FIG. 10: S5).

The sub CPU 64 of the controller 63 detects the pressure in the pressure cuffs 30a, 30b by the second pressure sensor 73, and determines whether the pressure cuffs 30a, 30b have reached a predetermined pressure threshold (FIG. 10: S6). The sub CPU 64 of the controller 63 drives the pump 71 until the pressure in the pressure cuffs 30a, 30b reaches a predetermined pressure threshold (FIG. 10: S5; NO), and continues the blood pressure calculation. When the sub CPU 64 of the controller 63 functioning as the pressure cuff controller 63A determines that the pressure in the pressure cuffs 30a, 30b has reached the predetermined pressure threshold (FIG. 10: S6; YES), the processing at the end of the blood pressure measurement is performed.

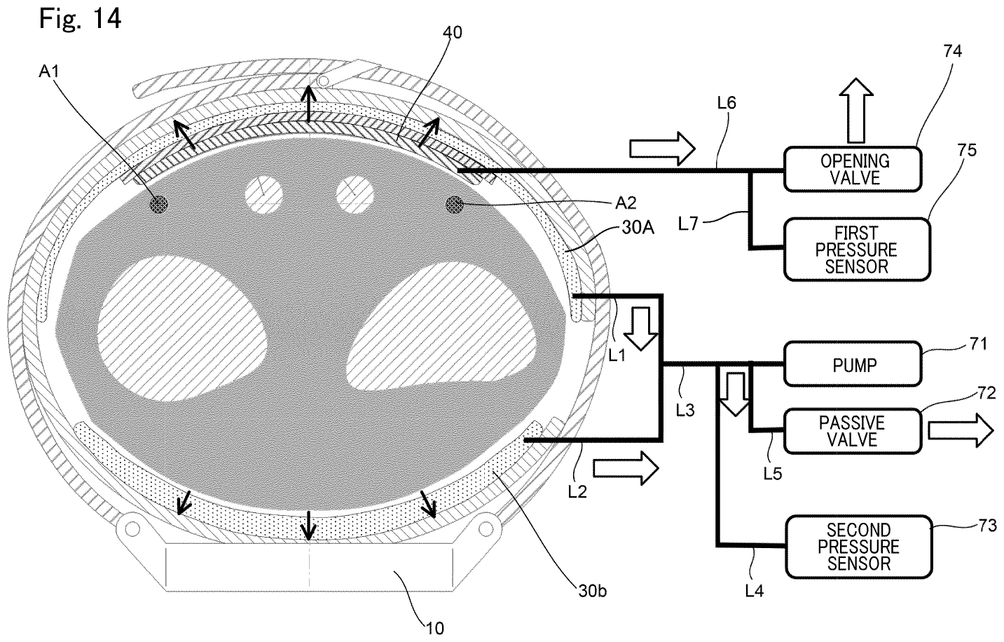

In the processing at the end of the blood pressure measurement, as illustrated in FIG. 10, the sub CPU 64 of the controller 63 functioning as the pressure cuff controller 63A turns off the pump 71 (FIG. 10: S7). As a result, as indicated by a white arrow in FIG. 14, the air in the pressure cuffs 30a, 30b is discharged via the passive valve 72 through the flow paths L1, L2, and L3, and the pressure cuffs are gradually decompressed. As described above, the sub CPU 64 of the controller 63 functioning as the pressure cuff controller 63A discharges the air as the fluid from the pressure cuffs 30a, 30b, and controls the pressure cuffs 30a, 30b so as to achieve a release state in which the compression of the human body (wrist) as the measurement site via the pressure cuffs 30a, 30b is released.

On the other hand, the sub CPU 64 of the controller 63 functions as the opening valve controller 63B, sets the opening valve 74 to the OFF state, and sets the opening valve 74 to the open state (FIG. 10: S8). As a result, the air in the sensing cuff 40 is discharged from the opening valve 74 as indicated by a white arrow in FIG. 14.

In the present embodiment, blood pressure measurement is performed by the constant volume sensing method as described above.

As described above, in the blood-pressure gauge 100 of the present embodiment, the sensing cuff 40 is not adjusted to the predetermined volume by the supply of the air from the pump 71, but the sensing cuff 40 is released to the atmospheric pressure by opening the opening valve 74, and the sensing cuff 40 is set to the predetermined volume by the restoring force of the elastic member 41 and then by closing the opening valve 74.

As a result, in the present embodiment, it is not necessary to route the flow path, which is the air path from the pump 71 built in the main body 10, to the sensing cuff 40 side, and it is not necessary to dispose the pressure sensor for the sensing cuff 40 on the pump 71 side. Therefore, it is not necessary to route the flow path, which is the air path for the pressure sensor for the sensing cuff 40. Therefore, it is possible to simplify the routing of the flow path.

In addition, since the opening and closing of the sensing cuff 40 are configured by the switching valve of only the off state and the on state of the opening valve 74, it is not necessary to dispose the flow path, which is the air path from the pump 71 side to the sensing cuff 40 side, and the routing of the flow path can be simplified.

The sub CPU 64 controls the pump 71 and the opening valve 74, detects the pressure in the pressure cuffs 30a, 30b using the second pressure sensor 73, and detects the pressure in the sensing cuff 40 using the first pressure sensor 75. The opening valve 74, the first pressure sensor 75, and the sub CPU 64 are configured as a substrate integrated type integrally mounted on the substrate 52, and the sub CPU 64 and the pump 71 and the second pressure sensor 73 on the fluid circuit LC1 side can be electrically connected by a simple wiring. Therefore, in the present embodiment, it is not necessary to perform complicated wiring not only for routing the flow path which is the air path but also for electrical connection.

In the present embodiment, a solenoid type valve is used as the opening valve 74, but the present invention is not limited to such an aspect, and a valve configured as a capacitance type or the like may be used as the opening valve 74.

In the above embodiment, the controller 63 includes the sub CPU 64 and the main CPU 65, but the controller 63 may include only the main CPU 65. In addition, although the controller 63 includes a CPU, the present invention is not limited thereto. The controller 63 may include a logic circuit (integrated circuit) such as a programmable logic device (PLD) or a field programmable gate array (FPGA).

### (Modification)

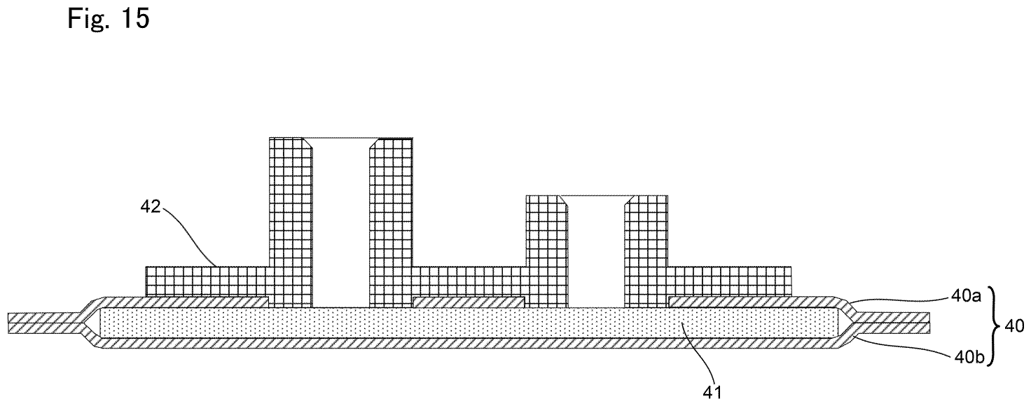

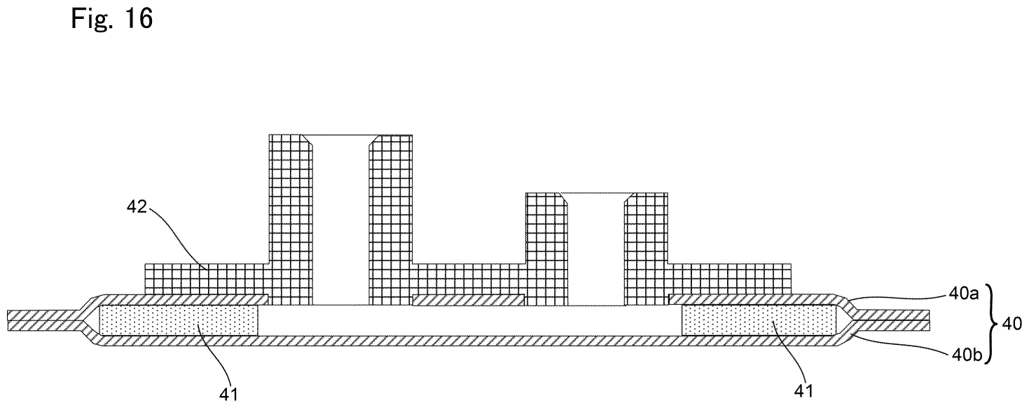

Next, a modification of the present embodiment will be described with reference to FIGS. 15 and 16. FIGS. 15 and 16 are cross-sectional views of a sensing cuff, a nipple portion, and an elastic member in the modification. In the embodiment described above, the aspect in which the nipple portion 42 is attached inside the sensing cuff 40 has been described. However, the present invention is not limited to such an aspect. For example, as illustrated in FIG. 15, the nipple portion 42 may be attached to the outside of the sensing cuff 40.

Further, in the embodiment described above, an aspect has been described in which the elastic member 41 made of a sponge having an open-cell structure is accommodated inside the sensing cuff 40 so as to uniformly fill the inside with almost no gap. However, the present invention is not limited to such an aspect. For example, as illustrated in FIG. 16, the elastic member 41 made of a sponge having an open-cell structure may be accommodated only at both ends in the short direction of sensing.

Also in these modifications, the sensing cuff 40 can be made to have a predetermined volume by releasing the pressure in the sensing cuff 40 to the atmospheric pressure by opening the opening valve 74, by the restoring force of the elastic member 41, and then by closing the opening valve 74.

### Second Embodiment

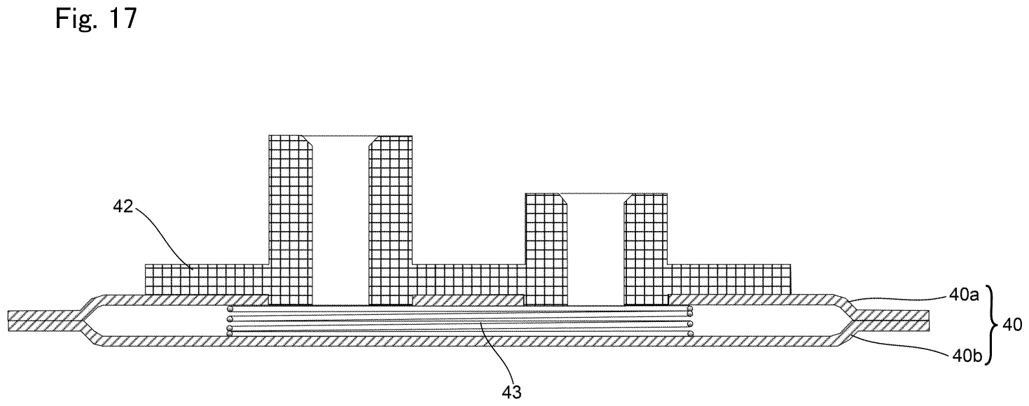

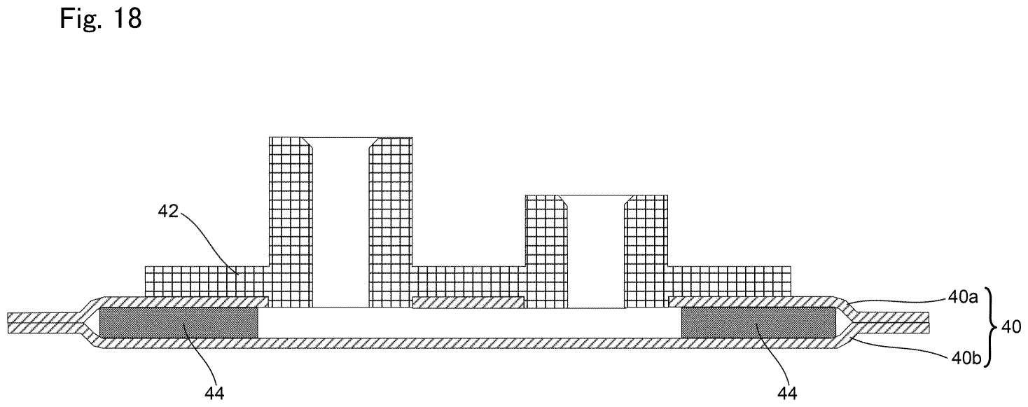

Next, a second embodiment of the present invention will be described with reference to FIGS. 17 and 18. FIGS. 17 and 18 are cross-sectional views of the sensing cuff, the nipple portion, and the elastic member according to the second embodiment. In the first embodiment, an elastic member made of a sponge having an open-cell structure is used as the elastic member. However, in the second embodiment, as illustrated in FIG. 17, a coil spring 43 is used as the elastic member. The coil spring 43 has a circular shape in a top view, and a plurality of coil springs 43 are arranged in the longitudinal direction of the sensing cuff 40. According to such an aspect as well, it is possible to realize the sensing cuff 40 having the predetermined volume by releasing the pressure in the sensing cuff 40 to the atmospheric pressure by opening the opening valve 74, by the restoring force of the coil spring 43, and then by closing the opening valve 74.

In the second embodiment, as illustrated in FIG. 18, a sheet 44 made of polyurethane (PU), polyethylene terephthalate (PET), or the like may be accommodated as a spacer at both ends of the sensing cuff 40 in the short direction. By applying tension to the first sheet 40a and the second sheet 40b having elasticity, it is possible to make the volume of the sensing cuff 40 constant and to impart restorability.

The above embodiments are merely examples, and various modifications can be made without departing from the scope of the present invention. The plurality of embodiments described above can be established independently, but combinations of the embodiments are also possible. In addition, various features in different embodiments can be established independently, but combinations of the features in different embodiments are also possible.

As described above, a blood-pressure gauge of this disclosure includes:
a pump;
a bag-shaped pressure cuff that is connected to the pump, and extends along a circumferential direction of a measurement site in order to received supply of a pressurizing fluid from the pump;
a sensing cuff that is not connected to the pump, includes a first sheet disposed to face an inner peripheral surface of the pressure cuff and a second sheet facing the first sheet, is configured in a bag shape, and extends in a circumferential direction so as to cross an artery passing portion of the measurement site;
a back plate that is interposed between the pressure cuff and the sensing cuff, extends along a circumferential direction of the measurement site, and transmits a pressing force from the pressure cuff to the sensing cuff;
an opening valve that is connected to the sensing cuff and is in either an open state in which an inside of the sensing cuff is conducted to outside air or a closed state in which the inside of the sensing cuff is not conducted to the outside air;
a pressure cuff controller that controls the pressure cuff to either a compression state in which a fluid is supplied to the pressure cuff and the measurement site is compressed via the pressure cuff or a release state in which the fluid is discharged from the pressure cuff and the compression of the measurement site via the pressure cuff is released;
an opening valve controller that controls the opening valve to be in either the open state or the closed state; and
a blood pressure calculator that calculates a blood pressure on a basis of a pressure change of the sensing cuff when the opening valve is in the closed state, in which the sensing cuff has restorability to make a volume in the sensing cuff a predetermined volume when the opening valve is in the open state.

The “fluid” is typically air, but may be other gases or liquids.

The “inner peripheral side” of the pressure cuff refers to a side facing the measurement site in an attachment state surrounding the measurement site.

In the blood-pressure gauge of the present disclosure, the inside of the sensing cuff is conducted to the outside air when the opening valve is in the open state, and is not conducted to the outside air when the opening valve is in the closed state. The sensing cuff has restorability so that the volume in the sensing cuff is a predetermined volume when the opening valve is in the open state. Therefore, the volume in the sensing cuff can be set to a predetermined volume only by opening the opening valve, restoring the sensing cuff, and closing the opening valve without routing the flow path from the means such as the pump that supplies the fluid to the sensing cuff from the pump side to the sensing cuff side. Thereafter, the pressure cuff controller supplies the fluid to the pressure cuff to bring the pressure cuff into the compression state of compressing the measurement site via the pressure cuff, and when the opening valve is in the closed state, the blood pressure is calculated based on the pressure change of the sensing cuff. Therefore, the air path connecting from the pump side to the sensing cuff side becomes unnecessary, and the routing of the air path connected to the sensing cuff can be simplified.

In the blood-pressure gauge of one embodiment, an elastic member is provided in the sensing cuff.

In the blood-pressure gauge according to this embodiment, when the opening valve is opened, the elastic member provided in the sensing cuff restores the sensing cuff, and the volume in the sensing cuff can be set to a predetermined volume only by closing the opening valve. Thereafter, the pressure cuff controller supplies the fluid to the pressure cuff to bring the pressure cuff into the compression state of compressing the measurement site via the pressure cuff, and when the opening valve is in the closed state, the blood pressure is calculated based on the pressure change of the sensing cuff. Therefore, the air path connecting from the pump side to the sensing cuff side becomes unnecessary, and the routing of the air path connected to the sensing cuff can be simplified.

In the blood-pressure gauge according to an embodiment, the elastic member is a sponge having an open-cell structure.

In the blood-pressure gauge according to this embodiment, the elastic member of the sponge having the open-cell structure provided in the sensing cuff is pressed and compressed at the time of blood pressure measurement to enable pulse pressure measurement by the sensing cuff. However, when the pressure in the sensing cuff is released to the atmospheric pressure before or after the blood pressure measurement, the sponge having the open-cell structure again contains air and is restored, and the volume of the sensing cuff can be made constant. Thereafter, the pressure cuff controller supplies the fluid to the pressure cuff to bring the pressure cuff into the compression state of compressing the measurement site via the pressure cuff, and when the opening valve is in the closed state, the blood pressure is calculated based on the pressure change of the sensing cuff. Therefore, the air path connecting from the pump side to the sensing cuff side becomes unnecessary, and the routing of the air path connected to the sensing cuff can be simplified.

In the blood-pressure gauge of one embodiment, the elastic member is a coil spring.

In the blood-pressure gauge according to this embodiment, the elastic member of the coil spring provided in the sensing cuff is pressed and compressed at the time of blood pressure measurement to enable pulse pressure measurement by the sensing cuff. However, when the pressure in the sensing cuff is released to the atmospheric pressure before or after the blood pressure measurement, the coil spring is restored, and the volume of the sensing cuff can be set to a constant volume. Thereafter, the pressure cuff controller supplies the fluid to the pressure cuff to bring the pressure cuff into the compression state of compressing the measurement site via the pressure cuff, and when the opening valve is in the closed state, the blood pressure is calculated based on the pressure change of the sensing cuff. Therefore, the air path connecting from the pump side to the sensing cuff side becomes unnecessary, and the routing of the air path connected to the sensing cuff can be simplified.

In the blood-pressure gauge of one embodiment, a spacer is provided in the sensing cuff.

In the blood-pressure gauge according to this embodiment, a sheet is accommodated in the sensing cuff as a spacer, but the volume of the sensing cuff can be set to a constant volume and the sensing cuff can have restorability by applying tension to a first sheet and a second sheet having elasticity. Therefore, when the pressure in the sensing cuff is released to the atmospheric pressure before or after the blood pressure measurement, the sensing cuff is restored, and the volume of the sensing cuff can be made constant. Thereafter, the pressure cuff controller supplies the fluid to the pressure cuff to bring the pressure cuff into the compression state of compressing the measurement site via the pressure cuff, and when the opening valve is in the closed state, the blood pressure is calculated based on the pressure change of the sensing cuff. Therefore, the air path connecting from the pump side to the sensing cuff side becomes unnecessary, and the routing of the air path connected to the sensing cuff can be simplified.

In the blood-pressure gauge of one embodiment,
in a preparation stage before the blood pressure is calculated by the blood pressure calculator, the pressure cuff and the sensing cuff is in an attachment state in which the pressure cuff and the sensing cuff are attached to the measurement site, the pressure cuff controller controls the pressure cuff to the release state, and the opening valve controller brings the opening valve into the closed state after bringing the opening valve into the open state, and
in a measurement stage in which the blood pressure is calculated by the blood pressure calculator, the pressure cuff and the sensing cuff is in the attachment state, the pressure cuff controller controls the pressure cuff to be in the compression state, and the blood pressure calculator calculates a blood pressure on a basis of a pressure change of the sensing cuff.

In the blood-pressure gauge of this embodiment, in the preparation stage before the blood pressure calculator calculates the blood pressure, the pressure cuff controller controls the pressure cuff to the release state in the attachment state in which the pressure cuff and the sensing cuff are attached to the measurement site. The opening valve controller opens the opening valve and then closes the opening valve. Therefore, even if the fluid is not supplied from the pump or the like, the sensing cuff has a predetermined volume. Then, in the measurement stage where the blood pressure is calculated by the blood pressure calculator, the pressure cuff controller controls the pressure cuff to be in the compression state in the attachment state. As a result, the sensing cuff is compressed against the measurement site, and the blood pressure calculator can calculate the blood pressure on the basis of the pressure change of the sensing cuff. As described above, the air path connecting from the pump side to the sensing cuff side becomes unnecessary, and the blood pressure can be accurately measured in the blood-pressure gauge capable of simplifying the routing of the air path connected to the sensing cuff.

In the blood-pressure gauge of one embodiment,
the opening valve is a valve that is brought into the open state or the closed state by bringing the opening valve into an off state or an on state.

In the blood-pressure gauge of this embodiment, the opening valve is turned into the open state by turning off the opening valve, and is turned into the closed state by turning on the opening valve. Therefore, the air path connecting from the pump side to the sensing cuff side becomes unnecessary, and the routing of the air path connected to the sensing cuff can be simplified.

The blood-pressure gauge of one embodiment, further includes:
a pressure sensor that detects a pressure of the sensing cuff; and
a substrate provided between the pressure cuff and the sensing cuff, in which
the opening valve, the pressure sensor, the opening valve controller, and the blood pressure calculator are integrally mounted on the substrate.

In the blood-pressure gauge of this embodiment, since the opening valve, the pressure sensor, the opening valve controller, and the blood pressure calculator are integrally mounted on a substrate provided between the pressure cuff and the sensing cuff, a flow path of the opening valve, the pressure sensor, and the sensing cuff can also be simplified, and wiring between the opening valve controller and the opening valve, and wiring between the pressure sensor and the blood pressure calculator can also be simplified.

In the blood-pressure gauge of one embodiment,
the opening valve is a solenoid type valve or a capacitance type valve.

In the blood-pressure gauge of this embodiment, the opening valve is a solenoid type valve or a capacitance type valve, so that it is possible to simplify the routing of the air path connecting from the pump side to the sensing cuff side.

The blood-pressure gauge of this embodiment further comprising:
a pressure sensor that detects a pressure of the sensing cuff; and
a substrate provided between the pressure cuff and the sensing cuff, in which
the opening valve, the pressure sensor, and the opening valve controller are integrally mounted on the substrate.

In the blood-pressure gauge of this embodiment, since the opening valve, the pressure sensor, and the opening valve controller are integrally mounted on a substrate provided between the pressure cuff and the sensing cuff, a flow path of the opening valve, the pressure sensor, and the sensing cuff can also be simplified, and wiring between the opening valve controller and the opening valve, and wiring between the pressure sensor and the blood pressure calculator can also be simplified.

As is clear from the above, the blood-pressure gauge of the present disclosure can simplify the routing of the air path connected to the sensing cuff.

What is claimed is:

1. A blood-pressure gauge comprising:
a pump;
a bag-shaped pressure cuff that is connected to the pump, and extends along a circumferential direction of a measurement site in order to received supply of a pressurizing fluid from the pump;
a sensing cuff that is not connected to the pump, includes a first sheet disposed to face an inner peripheral surface of the pressure cuff and a second sheet facing the first sheet, is configured in a bag shape, and extends in a circumferential direction so as to cross an artery passing portion of the measurement site;
a back plate that is interposed between the pressure cuff and the sensing cuff, extends along a circumferential direction of the measurement site, and transmits a pressing force from the pressure cuff to the sensing cuff;
an opening valve that is connected to the sensing cuff and is in either an open state in which an inside of the sensing cuff is conducted to outside air or a closed state in which the inside of the sensing cuff is not conducted to the outside air;
a pressure cuff controller that controls the pressure cuff to either a compression state in which a fluid is supplied to the pressure cuff and the measurement site is compressed via the pressure cuff or a release state in which the fluid is discharged from the pressure cuff and the compression of the measurement site via the pressure cuff is released;
an opening valve controller that controls the opening valve to be in either the open state or the closed state; and
a blood pressure calculator that calculates a blood pressure on a basis of a pressure change of the sensing cuff when the opening valve is in the closed state,
wherein the sensing cuff has restorability to make a volume in the sensing cuff a predetermined volume when the opening valve is in the open state.

2. The blood-pressure gauge according to claim 1, further comprising an elastic member in the sensing cuff.

3. The blood-pressure gauge according to claim 2, wherein the elastic member is a sponge having an open-cell structure.

4. The blood-pressure gauge according to claim 2, wherein the elastic member is a coil spring.

5. The blood-pressure gauge according to claim 1, further comprising a spacer in the sensing cuff.

6. The blood-pressure gauge according to claim 1, wherein
in a preparation stage before the blood pressure is calculated by the blood pressure calculator, the pressure cuff and the sensing cuff is in an attachment state in which the pressure cuff and the sensing cuff are attached to the measurement site, the pressure cuff controller controls the pressure cuff to the release state, and the opening valve controller brings the opening valve into the closed state after bringing the opening valve into the open state, and
in a measurement stage in which the blood pressure is calculated by the blood pressure calculator, the pressure cuff and the sensing cuff is in the attachment state, the pressure cuff controller controls the pressure cuff to be in the compression state, and the blood pressure calculator calculates a blood pressure on a basis of a pressure change of the sensing cuff.

7. The blood-pressure gauge according to claim 1, wherein the opening valve is a valve that is brought into the open state or the closed state by bringing the opening valve into an off state or an on state.

8. The blood-pressure gauge according to claim 1, further comprising:
a pressure sensor that detects a pressure of the sensing cuff; and
a substrate provided between the pressure cuff and the sensing cuff,
wherein the opening valve, the pressure sensor, the opening valve controller, and the blood pressure calculator are integrally mounted on the substrate.

9. The blood-pressure gauge according to claim 1, wherein the opening valve is a solenoid type valve or a capacitance type valve.

10. The blood-pressure gauge according to claim 1 further comprising:
a pressure sensor that detects a pressure of the sensing cuff; and
a substrate provided between the pressure cuff and the sensing cuff,
wherein the opening valve, the pressure sensor, and the opening valve controller are integrally mounted on the substrate.
